(19) United States
(12) Patent Application Publication
(10) Pub. No.: US 2018/0224423 A1
(43) Pub. Date: Aug. 9, 2018

(54) NEURON INHIBITION BY INFRARED LIGHT

(71) Applicant: Government of the United States as Represented by the Secretary of the Air Force, Wright-Patterson AFB, OH (US)

(72) Inventors: Alexandra J. Walsh, San Antonio, TX (US); Hope T. Beier, San Antonio, TX (US); Bennett L. Ibey, San Antonio, TX (US)

(73) Assignee: Government of the United States as Represented by the Secretary of the Air Force, Wright-Patterson AFB, OH (US)

(21) Appl. No.: 15/891,700

(22) Filed: Feb. 8, 2018

Related U.S. Application Data
(60) Provisional application No. 62/456,238, filed on Feb. 8, 2017.

(51) Int. Cl. G01N 33/487

(52) U.S. Cl. CPC G01N 33/48728; G01R 19/2506

(57) ABSTRACT

A method of inhibiting excitable cells. The method includes exposing the excitable cells to a pulse of infrared light having a wavelength ranging from 700 nm to about 3 μm and having a radiant exposure at a surface of the excitable cells ranging from 1 μJ/cm² to 1000 J/cm².

Pursuant to 37 C.F.R. § 1.78(a)(4), this application claims the benefit of and priority to prior filed co-pending Provisional Application Ser. No. 62/456,238, filed Feb. 8, 2017, the disclosure of which is expressly incorporated herein by reference, in its entirety.

### RIGHTS OF THE GOVERNMENT

The invention described herein may be manufactured and used by or for the Government of the United States for all governmental purposes without the payment of any royalty.

### FIELD OF THE INVENTION

The present invention relates generally to neuron inhibition and, more particularly, to methods and devices for neuron inhibition.

### BACKGROUND OF THE INVENTION

Excitable cells, such as neurons, conduct signals throughout the body by membrane depolarization and action potentials. Many techniques, including electrode-based electrophysiology, optogenetics, and infrared light neuron stimulation, exist for inducing action potentials within neurons. However, methods for inhibiting neuronal activity remain limited. A conventional approach to action potential inhibition has been constant electrical stimulation; however, this method requires large electric fields, invasive contact by electrodes, and cannot be spatially confined to a small region (such as a single neuron). Another approach to neuron inhibition has been through specific optogenetic proteins, which hyperpolarize the cell upon activation with light. Optogenetic solutions, unfortunately, require transfection of a foreign, bacterial protein and, thus, remain impractical for human use.

Direct inhibition of action potentials in excitable cells would be useful for a variety of medical conditions that are characterized by over-active neurons: for example, pain, epilepsy, and arrhythmias. Thus, it would be of great advantage to develop a method of blocking action potential generation either at the generation source or along the action potential propagation to reduce the undesired effect.

As a result, there remains a need for devices and methods of neuron inhibition.

### SUMMARY OF THE INVENTION

The present invention overcomes the foregoing problems and other shortcomings, drawbacks, and challenges of inhibiting neurons. While the invention will be described in connection with certain embodiments, it will be understood that the invention is not limited to these embodiments. To the contrary, this invention includes all alternatives, modifications, and equivalents as may be included within the spirit and scope of the present invention.

According to an embodiment of the present invention, a method of inhibiting excitable cells includes exposing the excitable cells to a pulse of infrared light having a wavelength ranging from 700 nm to about 3 μm and having a radiant exposure at a surface of the excitable cells ranging from 1 μJ/cm² to 1000 J/cm².

Other embodiments of the present invention include a system that is configured to inhibit action potentials in excitable cells. The system includes a radiant source configured to emit infrared light. At least one timer is configured to design pulses of the infrared light according to a desired pulse sequence, and a delivery system is configured to direct the pulses of infrared light to the excitable cells. A control is configured to control the radiant source, the at one timer, the at least one delivery system, or a combination thereof.

Still other embodiments of the present invention include a method of inhibiting action potentials in excitable cells by exposing the excitable cells to a pulse of infrared light. The infrared light has a wavelength ranging from 700 nm to about 3 μm and a radiant exposure that is configured to produce a thermal rise of at least 1° C./ms proximate to a surface of the excitable cell.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flow chart illustrating a method of inhibiting neurons by infrared light according to an embodiment of the present invention.

FIG. 2 is a schematic of a fluorescence microscope for applying infrared light to neurons according to an embodiment of the present invention.

FIG. 2A is an enlargement of the portion enclosed in box 2A in FIG. 2.

FIG. 3 is an exemplary pulse sequence for applying infrared light and for use with embodiment of the present invention.

FIG. 4 is a schematic illustration of a computer suitable for use with one of more embodiments of the present invention.

FIG. 5 is a schematic illustration of a neuron.

FIG. 5A is an enlargement of the portion enclosed with box 5A in FIG. 5.

FIG. 6 is an exemplary fluorescence image illustrating successful transfection of an OptoPatch2 plasmid into a neuron.

FIG. 7 is an exemplary, low resolution, fluorescence image illustrating no photobleaching when the sample was exposed to constant illumination for up to 4.5 s.

FIG. 7A is a graphical representation of fluorescence of the neuron imaged in FIG. 7, over time.

FIG. 8 is an exemplary, low resolution, fluorescence image of the projection of QuasAr42 fluorescence from a neuron when exposed to pulsed, blue light configured to initiate action potentials.

FIGS. 8A and 8B are fluorescence intensity plots, over time, of the neuron imaged in FIG. 8, where spikes are action potentials.

FIG. 9 is an exemplary, low resolution, fluorescence image of the projection of QuasAr42 fluorescence from a neuron when exposed to light of the sequence illustrated in FIG. 10.

FIGS. 9A and 9B are fluorescence intensity plots, over time, demonstrating a rapid, short lived decrease in action potentials due to a thermal gradient. FIG. 9C is a fluorescence intensity plot demonstrating no effect or bleed-through from the infrared pulse in regions without neurons.

FIGS. 9D and 9E are fluorescence intensity plots exhibiting the recovery of action potentials after removing the infrared light.

FIG. 9F is a fluorescence intensity plot demonstrating no effect or bleed-through from the infrared pulse in regions without neurons.

FIG. 10 is a pulse sequence for infrared light exposing the neuron imaged in FIG. 9.

FIG. 11 is a graphical representation of a number of spikes observed per second for neurons exposed to 5 ms pulses of blue light.

FIGS. 12A-12C are fluorescence intensity plots of neurons exposed to tetrodotoxin.

FIG. 13 is a graphical illustration of short infrared laser pulse induced action potentials and the induced temperature increase at the cell soma.

FIGS. 14A-14C are representative QuasAr2 fluorescence spike plots demonstrating the uniformity of action potential spiking at 10 Hz.

FIGS. 15A-15C are representative QuasAr2 fluorescence spike demonstrating recovery of action potentials and reinstatement of the action potential block.

FIGS. 16A and 16B illustrate the length of action potential block with subsequent recovery to consistent action potential spikes that correlate with blue light stimulation.

FIGS. 17A and 17B graphically illustrate an average number of spikes per second following a short infrared laser pulse exposure.

FIG. 18 graphically illustrates the improved consistency of action potentials at 15 s and increased, inconsistently, with radiant exposure.

FIG. 19 is a representative QuasAr2 fluorescence spike before, during, and after a short infrared laser pulse exposure.

FIGS. 19A and 19B are enlargements of the respective and indicated portions in FIG. 19.

FIGS. 20A and 20B are histograms illustrating a number of action potentials spikes per second before, during, and for two time periods after exposure.

FIGS. 21A-21C graphically illustrate uniform action potential generation before the short infrared laser pulse, reduced action potential generation during exposure, and recovery after exposure terminated.

FIGS. 22A-22C graphically illustrate the reduced, average number of action potentials per second with greater exposure.

It should be understood that the appended drawings are not necessarily to scale, presenting a somewhat simplified representation of various features illustrative of the basic principles of the invention. The specific design feature of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

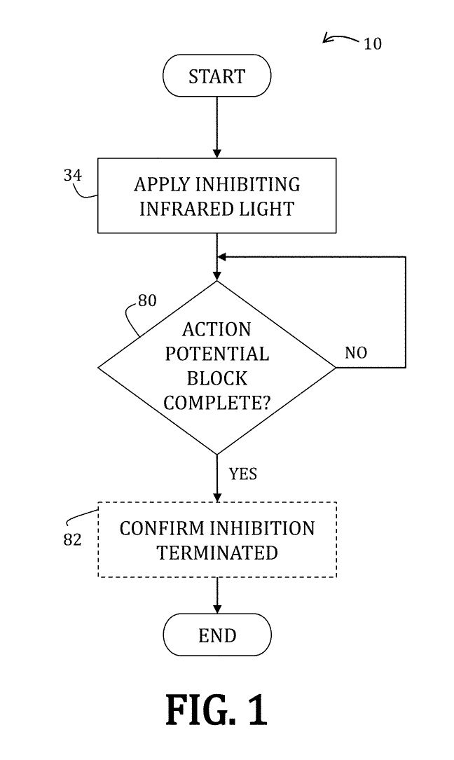

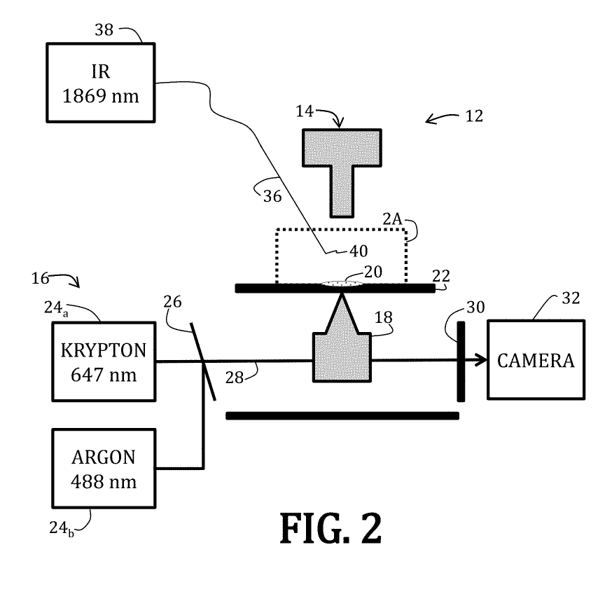

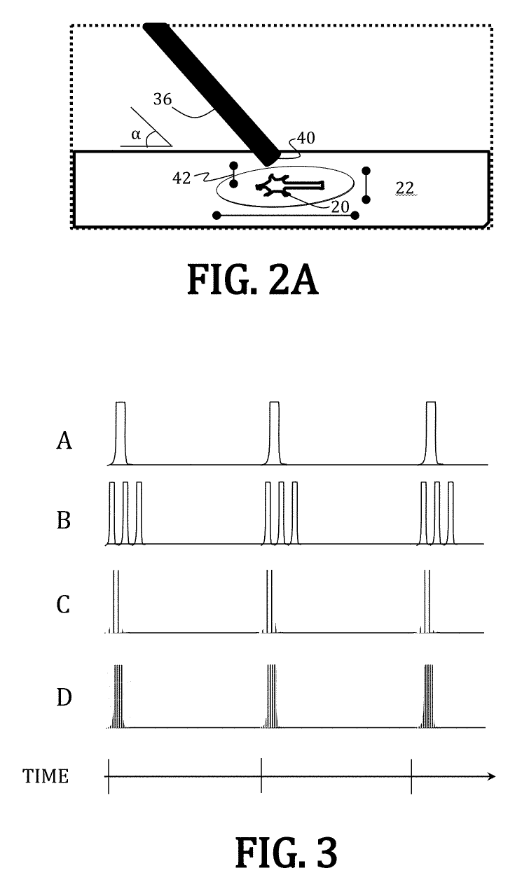

Turning now to the figures, and in particular to FIGS. 1 and 2, a flowchart 10 and exemplary system 12 for illustrating a method of using short infrared laser pulse neuron inhibition according to an embodiment of the present invention is described. The illustrated embodiment includes a fluorescence optical microscope 14, wherein some components of the optical microscope 14 are omitted for clarity and to better illustrate certain aspects of the invention. The optical microscope 14, as illustrated, comprises a light source 16 and at least one optic 18 having a lens therein (not shown). The light source 16 is configured to generate light to illuminate a biological sample 20 on or in a sample container (such as a glass slide) on a sample stage 22. The light source may be, for example, 25 mW of 568 nm light from an argon-krypton ion laser 24ₐ, 24_{b}, which is redirected by a dichroic mirror 26 to the optic 18, which is illustrated as an oil immersion objective (such as those from Olympus Corp., Shinjuku Japan). The emitted light (represented by arrow 28) from the laser 24ₐ, 24_{b} may be coupled to the sample and to a 647 nm long pass filter 30 and a camera 32. The camera 74, for example, an Electron Multiplying Charge Coupled Device camera (AndorTechnology Ltd., Belfast, Northern Ireland) detected emission light with a frame rate of 999 Hz. Generally, any suitable system may be used, including, for example, an inverted or upright microscope, an inspection microscope, a telescope, or any other suitable combination of a lens system, either through an optical fiber or directly from the laser, as described below.

Using the exemplary system 12 of FIG. 2, and referring still to FIG. 1, after the sample 20 is placed within the system 12, and at start, a short infrared pulse of light may be applied to the sample 20, which may be a neuron or other excitable tissues for which inhibition is desired (Block 34). The infrared pulse may be configured such that provides sufficient energy, based on the absorption of the pulse energy by fluid composing or surrounding the sample 20, to produce a thermal rise of at least 1° C./ms in proximity to the sample 20, whether single cell or tissue. A duration of the infrared pulse should be brief (less than about 10% duration) compared to a time of inhibition, that is, a desired time for which excitation of the sample 20 is inhibited.

The infrared pulse may be delivered, according to one embodiment of the present invention, by a delivery system, illustrated in FIG. 2 as an optic fiber 36. The infrared pulse may be obtained from an infrared light source 38, embodiments of which vary greatly. Some exemplary embodiments of infrared light sources may include diode or fiber-based laser systems (manufactures such as SemiNex Corp. (Peabody, Mass.) or IPG Photonics Corp. (Oxford, Mass.), broad-band infrared light sources, free-beam optics, fiber-optics, and digital micro-mirrors. Selection of the delivery system may depend, at least in part, on a desired target, which, again, may range from a single neuron (microscopic) to nerve bundles or tissues (macroscopic). An end 40 of the fiber 36 may be angled, a, with respect to and positioned above (line 42) a plane in which the sample 20 resides. For the illustrative embodiment, the end 40 is positioned with an a of about 45° and 500 μm above the sample.

The infrared light source 38 and delivery system (illustrated as the fiber optic 36) may further comprise any number of electronics and controllers necessary to supply a desired exposure of infrared light to the sample 20 or a desired region of interest and as would be understood by those of ordinary skill in the art having the benefit of the disclosure provided herein. One such electronic device may be a timer device coupled to the infrared light source 38 for manipulating the infrared light emitted according to a pulse sequence specifying, for example, a duration, a repetition, a duty cycle, a frequency, and so forth of the infrared pulse. Exemplary pulse sequences (A, B, C, and D) are illustrated in FIG. 3, wherein each sequence has similar timing but varying pulse structure, pulse number, duty cycle, and so forth. Although not specifically shown, the duration and repetition rate of the infrared pulses may also be varied according to a desired level of energy deposition. Generally, for most embodiments, the wavelength of the infrared pulse may range from about 700 nm to about 3 and the duration of the infrared pulse may range from a microsecond or a millisecond scale to about 100 ms. The pulse repetition frequency of the infrared pulse may range from about 100 Hz to about 0 Hz (that is, continuous exposure) or even include a single laser pulse.

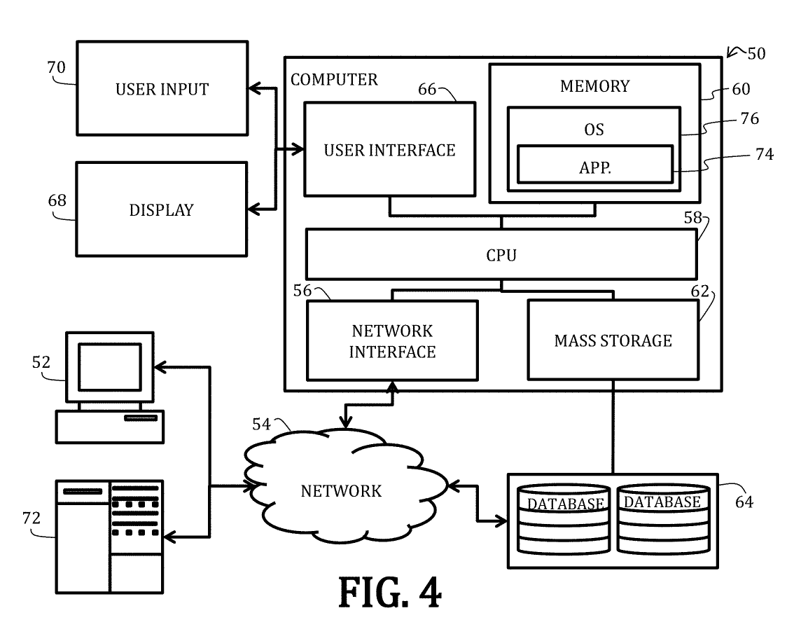

The controller and electronics may be operably coupled to a computer 50, which is described in greater detail with respect to FIG. 4, and which may be considered to represent any type of computer, computer system, computing system, server, disk array, or programmable device such as multi-user computers, single-user computers, handheld devices, networked devices, or embedded devices, etc. The computer 50 may be implemented with one or more networked computers 52 using one or more networks 54, e.g., in a cluster or other distributed computing system through a network interface (illustrated as “NETWORK I/F” 56). The computer 50 will be referred to as “computer” for brevity's sake, although it should be appreciated that the term “computing system” may also include other suitable programmable electronic devices consistent with embodiments of the invention.

The computer 50 typically includes at least one central processing unit (illustrated as “CPU” 58) coupled to a memory 60 along with several different types of peripheral devices, e.g., a mass storage device 62 with one or more databases 64, an input/output interface (illustrated as “User Interface” 66 with associated display 68 and user input devices 70), and the Network I/F 56. The memory 60 may include dynamic random access memory (“DRAM”), static random access memory (“SRAM”), non-volatile random access memory (“NVRAM”), persistent memory, flash memory, at least one hard disk drive, and/or another digital storage medium. The mass storage device 62 is typically at least one hard disk drive and may be located externally to the computer 50, such as in a separate enclosure or in one or more networked computers 52, one or more networked storage devices (including, for example, a tape or optical drive), and/or one or more other networked devices (including, for example, a server 72).

The CPU 58 may be, in various embodiments, a single-thread, multi-threaded, multi-core, and/or multi-element processing unit (not shown) as is well known in the art. In alternative embodiments, the computer 50 may include a plurality of processing units that may include single-thread processing units, multi-threaded processing units, multi-core processing units, multi-element processing units, and/or combinations thereof as is well known in the art. Similarly, the memory 60 may include one or more levels of data, instruction, and/or combination caches, with caches serving the individual processing unit or multiple processing units (not shown) as is well known in the art.

The memory 60 of the computer 50 may include one or more applications (illustrated as “APP.” 74), or other software program, which are configured to execute in combination with the Operating System (illustrated as “OS” 76) and automatically perform tasks necessary for operating the timers, infrared energy source, and so forth, with or without accessing further information or data from the database(s) 64 of the mass storage device 62.

Those skilled in the art will recognize that the environment illustrated in FIG. 4 is not intended to limit the present invention. Indeed, those skilled in the art will recognize that other alternative hardware and/or software environments may be used without departing from the scope of the invention.

Returning again to FIGS. 1 and 2, and after applying the inhibiting infrared pulse for a period of time (Block 34), it is determined whether inhibition is completed (Decision Block 80). If a need for inhibition continues, (“No” branch of decision block 80), then the method returns and awaits a subsequent determination. If inhibition is complete (“Yes” branch of decision block 80), then inhibition may end (Block 82). Optionally, termination of inhibition may end be observed, confirmed, or both by restoration of action potentials.

Although not specifically illustrated herein, it would be appreciated by those having ordinary skill in the art would readily appreciate that according to some embodiments of the present invention, an infrared recording device may be included and configured to record the infrared radiation effects on the target.

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

### Example 1

Additional information regarding experimentations provided herein may be found in A. J. WALSH et al., “All optical experimental design for neuron excitation, inhibition, and action potential detection,” Proc. SPIE, Vol. 9690 (2016) 6 pages total and A. J. WALSH et al., “Action potential block in neurons by infrared light,” Neurophotonics, Vol. 3 (2016) 040501. The disclosures of these non-patent literature documents are incorporated herein by reference, each in its entirety.

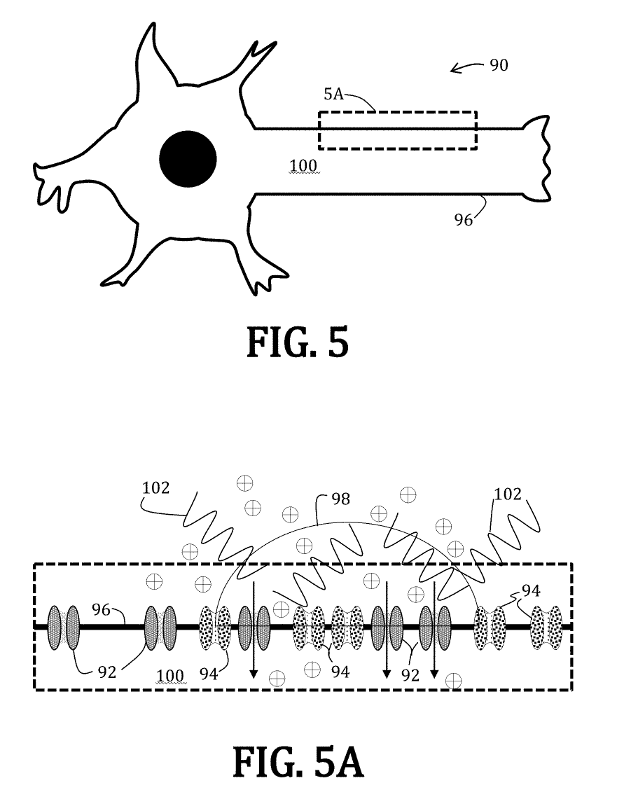

Neuron and glial cells 90 (one of which being schematically illustrated in FIGS. 5 and 5A) were isolated from neonatal rat hippocampi and were transfected with OptoPatch2 plasmid (plasmid no. 51694, Addgene, Cambridge Mass.) by an AMAXA NUCLEOFECTOR device (Lonza AG, Basel, Switzerland) according to methods understood by those have skill in the art. The OptoPatch2 plasmid causes the cell 90 to express a channelrhodopsin protein (“CheRiff” 92) and a fluorescent archaerhodopsin 3 protein (“Arch” 94) across a cell membrane 96, illustrated in FIG. 5A. CheRiff 52 is configured to absorb blue light (a bubble 98 of blue light illumination is illustrated) and thereby evoke an action potential in the cell 90. The expression of CheRiff 92 in the cells 90 was deliberate for controllable stimulation of action potentials. Arch 94 is configured to reports a change in membrane potential (localized influx of sodium and potassium ions (collectively illustrated as “⊕”) into the cytoplasm 100 through the cell membrane 96) via an increase in an intensity of fluorescence emission (illustrated as photon packets 102). The expression of Arch 54 in the cells 50 was deliberate for optically observing and recording action potential firing.

Seven days after transfection, cells 90 were incubated with 5 μM alltrans retinol for 30 min, washed with standard outside solution (2 mM MgCl₂, 5 mM KCl, 10 mM HEPES, 10 mM glucose, 2 mM CaCl₂, and 13.5 mM NaC, pH of 7, osmolarity of 300 mOsm), and imaged on a customized fluorescence microscope, such as the exemplary system 12 of FIG. 2.

The membrane protein, CheRiff 92, has peak absorption at 474 nm. Thus, to induce action potentials, the transfected cells 90 were illuminated with pulsed light from an infrared light source (not shown) (for example, a 470 nm, 50 mW, diode (Thorlabs Inc., Newton, N.J.) via a 200 μm fiber. The pulse rate was 5 Hz with a 10 ms pulse duration, which was generated by a digital delay generator (not shown) (Stanford Research Systems, Inc., Sunnyvale, Calif.).

Inhibition, according to the particular embodiment of the present invention, was accomplished by a system similar what is illustrated in FIG. 2. The infrared pulse of 1869 nm light was delivered by optic fiber 36, from an infrared light source 38 (Aculight laser) with a pulse duration of 4 ms. The end 40 of the fiber 36 was set at a of 45° and about 500 μm above the plane in which the cells 20 reside. A Stanford delay generator (not shown) initiated the infrared laser pulse and, 100 ms later, initiated a train of blue light pulses.

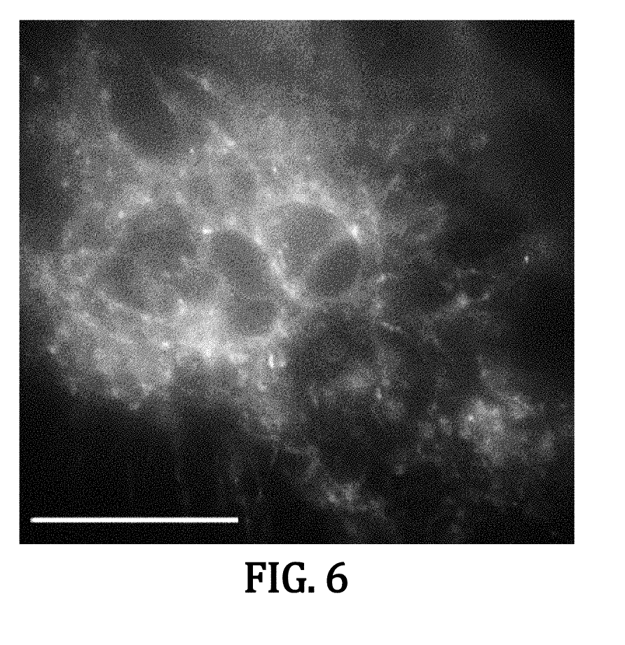

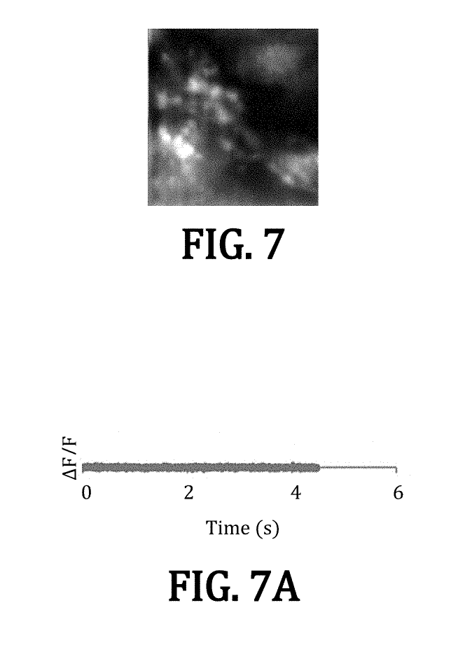

Successful transfection of OptoPatch2 plasmid was confirmed by imaging and excitation by 25 mW of 468 nm light. FIG. 6 includes an exemplary image of confirmed transfection. Further imaging with a limited field of view (“FOV”) (for example, FIG. 7 have a FOV of 92×92 pixels) permitted increased frame rates (for example, of up to 999 Hz). As shown in FIG. 7A, no photobleaching was observed for constant illumination of up to 4.5 s.

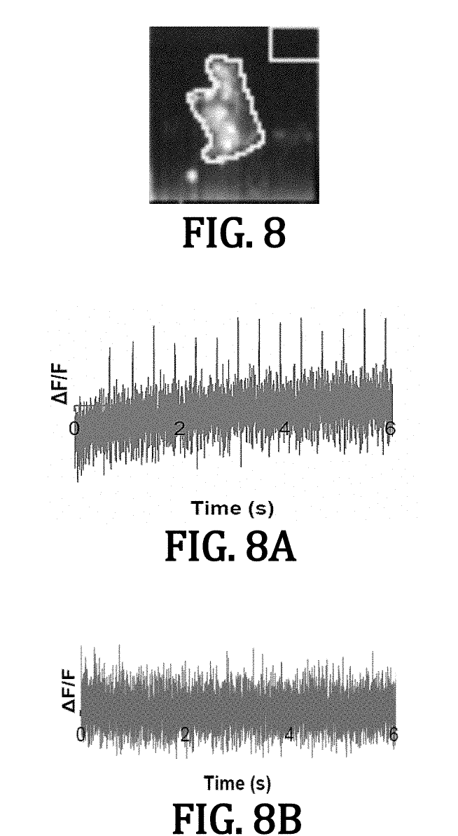

FIG. 8 is a limited FOV image of the projection of QuasAr2 fluorescence of an isolated neuron when the neuron was exposed to pulsed, blue light suitable for initiating action potentials. FIG. 8A is a graphical plot of fluorescence intensity over time with action potentials appearing as spikes. FIG. 8B includes a plot similar to that shown in FIG. 8A; however, in FIG. 8B the FOV is located over a background portion adjacent to the neuron. FIG. 8B does not include spikes, which suggests the spikes of FIG. 8A are associated with action potentials and not blue light leaking into the camera.

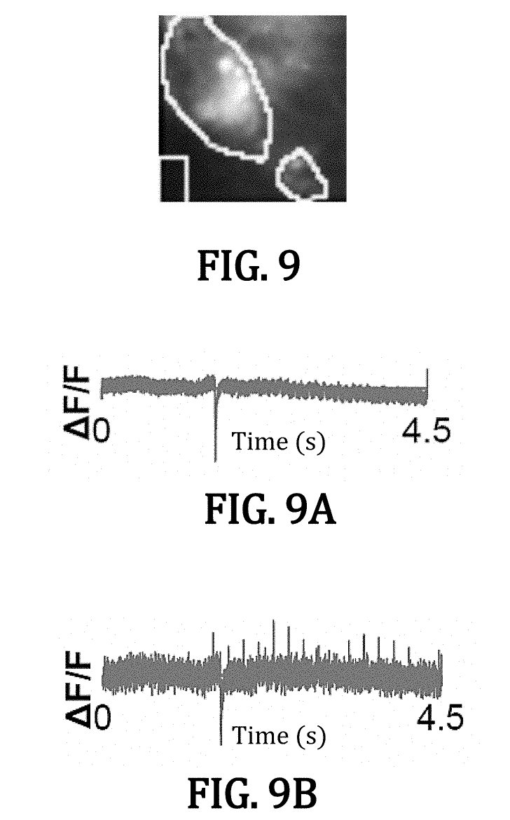

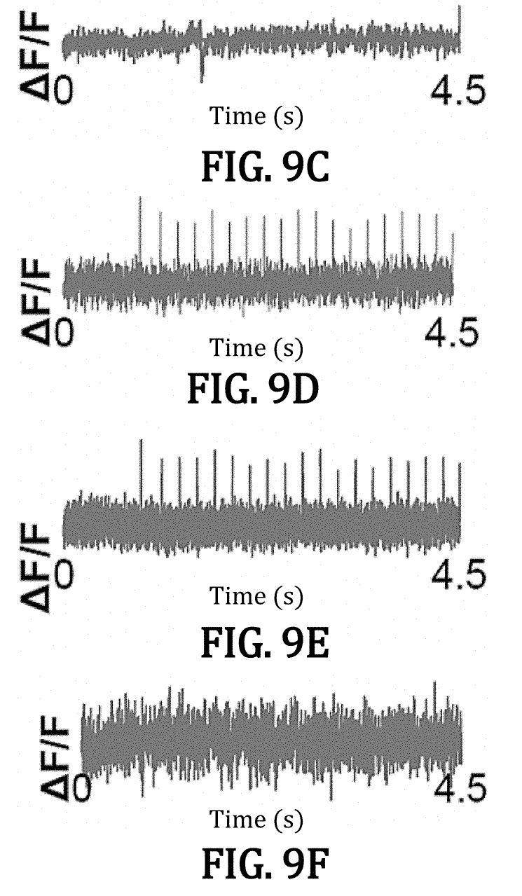

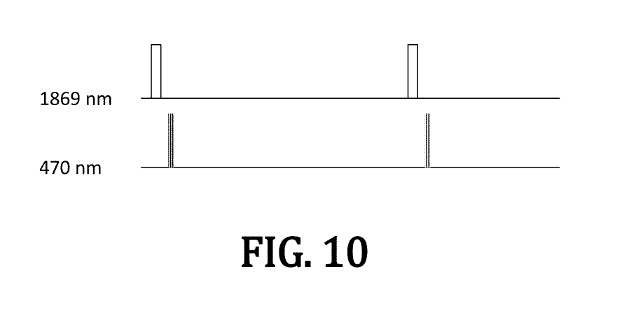

For testing inhibition, an infrared pulse (as described above) was delivered to a neuron, followed by (about 100 ms thereafter) a pulse of blue light configured to initiate an action potential, such as a 5 Hz blue light applied after a delay for a period of time. FIG. 9 is a limited FOV image of the projection of QuasAr2 fluorescence of an isolated neuron having the pulse sequence of FIG. 10 having 10 ms pulses for approximately 4 s, followed by 2 min of no illumination. The pulsed light was then repeated. The graphs of FIGS. 9A and 9B demonstrate a rapid, short-lived decrease in fluorescence in the imaged cell due to a thermal gradient and a change in the index of refraction caused by the infrared pulse. Complete action potential inhibition was observed for about 2.8 s following the infrared pulse (FIG. 9A). Action potential dampening was observed for a region of interest adjacent the neuron (FIG. 8B). FIG. 9C demonstrates no effect or bleed-through from the infrared pulse or the blue light in regions without neurons.

After a recovery period of approximately 2 min, the same FOV of FIG. 9A was reimaged and exposed to blue light. The neuron exhibited recovery of action potentials at both regions of interest (FIGS. 9D and 9E). Again, the adjacent spot failed to show spikes from the blue light, as shown in FIG. 9F.

### Example 2

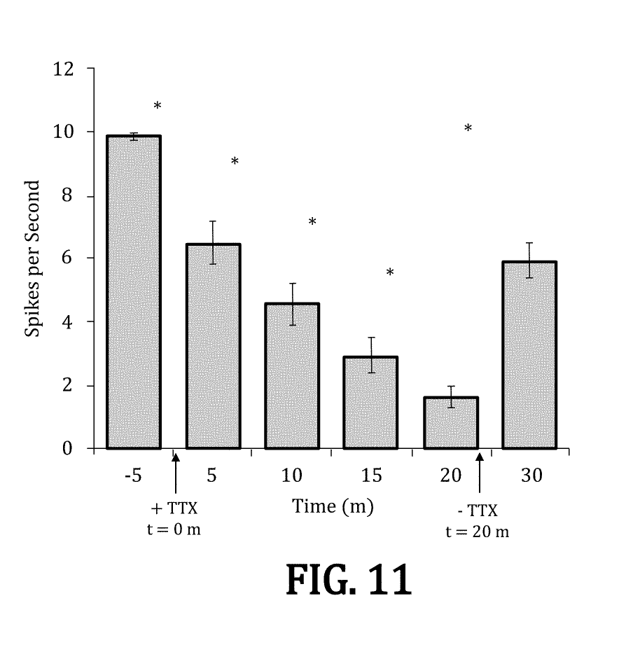

Optopatch2 plasmid was transfected into neurons dissociated from rat hippocampi. Arch fluorescence imaging was performed on a custom built wide-field fluorescence microscope with 647 nm illumination light (6.6 mW to 6.7 mW) provided by a krypton laser. CheRiff was activated with co-aligned 488 nm light from an argon laser (55 μW). Successful transfection was confirmed through observation of spikes in QuasAr2 fluorescence when neurons were exposed to 5 ms pulses of blue light (FIG. 11).

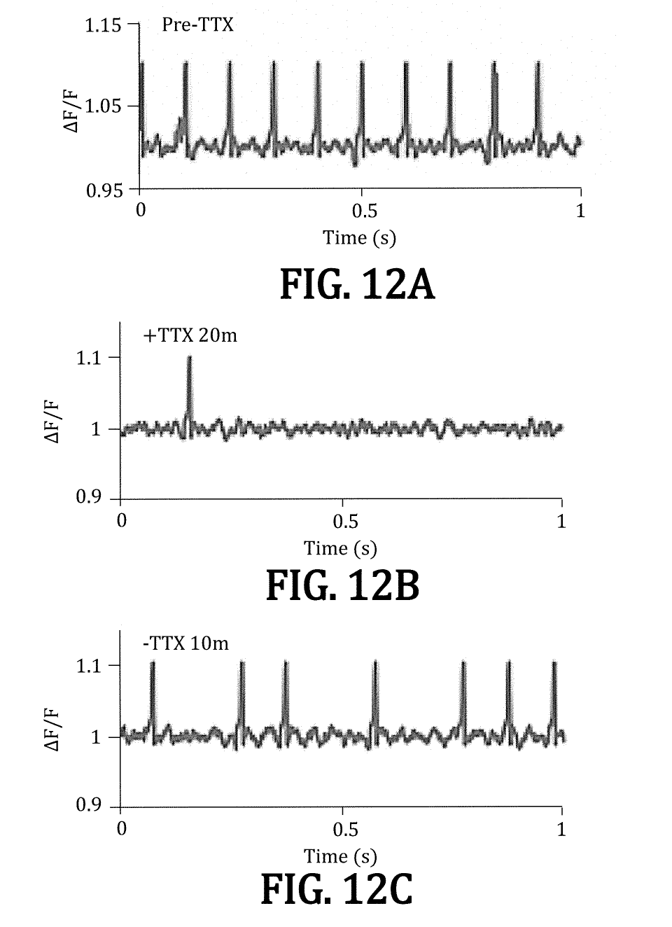

Exposing the neurons to tetrodotoxin (“TTX”), a potent neurotoxin, significantly reduced the frequency of spikes (p<0.0001) (FIGS. 12A-12C). Throughout the infrared exposure experiments, QuarsAr2 was continuously imaged as the neuron was exposed to 5 ms blue light pulsed at 10 Hz. No loss of action potential spikes in QuasAr2 fluorescence was observed for imaging sessions greater than 60 s.

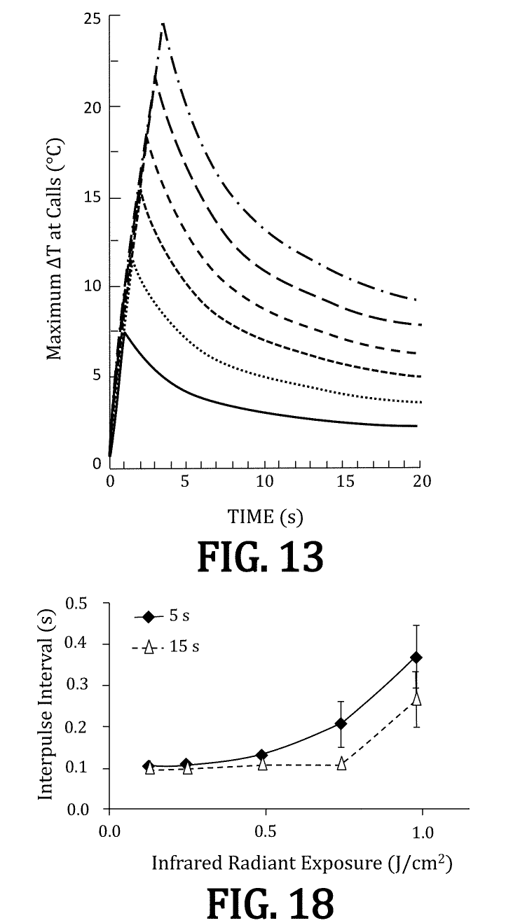

The ability to block action potentials was investigated by exposing the transfected neurons to 1869 nm laser pulses of varying exposures (0.13 J/cm² to 1.03 J/cm², achieved by varying the pulse length from 0.7 ms to 5.73 ms) by 200 μm fiber delivery. The fiber was position at a 45° angle with respect to, and about 215 μm diagonally above (185 μm vertically above), a plane in which the cells reside. Short infrared laser pulse induced action potential induced temperature increase at the cell soma was estimated to be between 3° C. and 25° C. (FIG. 13). Fiber delivery enables precise exposure of targeted neurons and reduced field effects.

TABLE 1

| Pulse Width | Energy | Radiant Exposure at Cells | ΔT |
|---|---|---|---|
| (ms) | (mJ) | (J/cm²) | (° C.) |
| 0.70 | 0.86 | 0.13 | 3.02 |
| 1.37 | 1.68 | 0.25 | 5.91 |
| 2.15 | 2.64 | 0.39 | 9.28 |
| 2.73 | 3.35 | 0.49 | 11.78 |
| 3.58 | 4.39 | 0.64 | 15.45 |
| 4.10 | 5.03 | 0.74 | 17.70 |
| 5.01 | 6.14 | 0.90 | 21.62 |

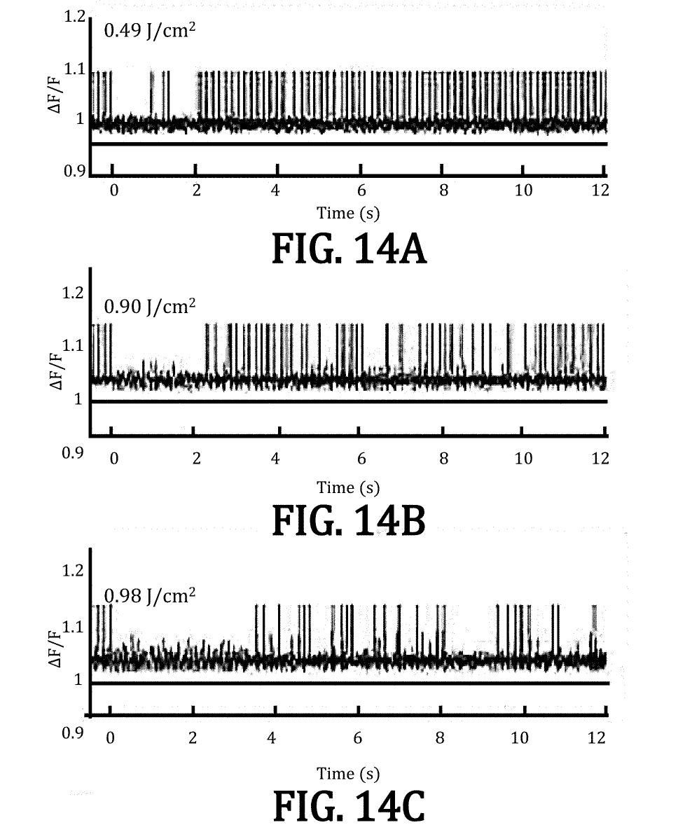

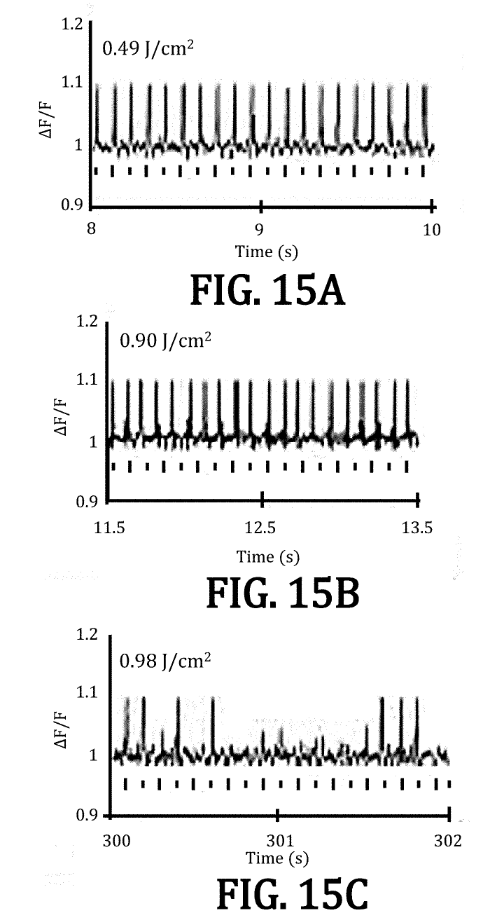

Full recovery of action potentials was observed following a short infrared laser pulse induced action potential block having radiant exposures of less than about 0.90 J/cm² (refer to Table 1, above). FIGS. 14A-14C are representative QuasAr2 fluorescence spike plots demonstrating the uniformity of AP spiking at 10 Hz (correlating to the pulsed blue light), the duration of the action potential block following 0.49 J/cm², 0.09 J/cm², and 0.98 J/cm² short infrared laser pulse exposure. FIGS. 15A and 15B demonstrate a robust recovery of uniform and consistent action potential following 0.49 J/cm² and 0.90 J/cm² exposures; FIG. 15C demonstrates action potential block induced by a 0.98 J/cm² short infrared laser pulse. No action potential was observed, even 5 min after the 0.98 J/cm² short infrared laser pulse, which suggests neural damage. These results suggest a threshold to recovery of action potential being between 0.9 J/cm² and 0.98 J/cm².

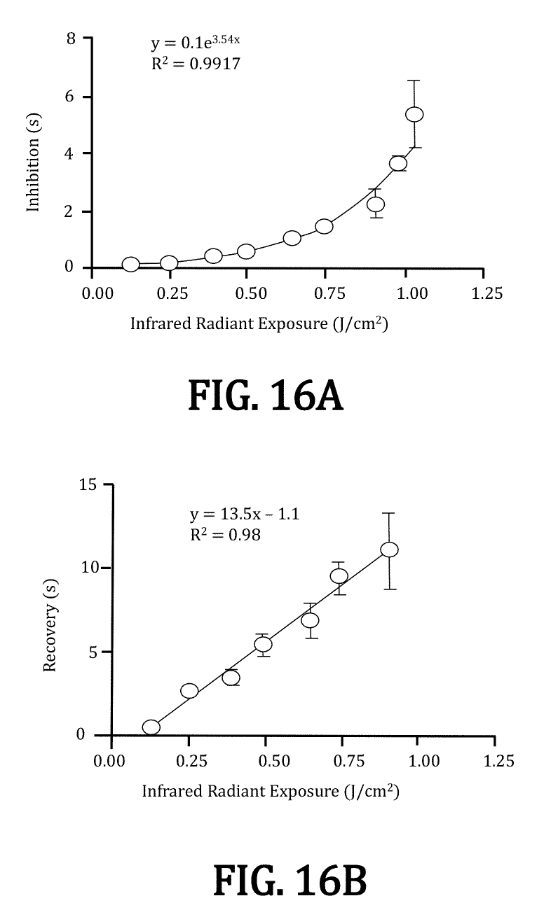

FIGS. 16A and 16B illustrate the length of action potential block with subsequent recovery to consistent action potential spikes that correlate with blue light stimulation scaled with short infrared laser pulse irradiance. The action potential block persisted for about 0.26 s with a 0.25 J/cm² exposure (p<0.0001 versus 0 s) or up to 5.37 s with a 1.03 J/cm² exposure (p<0.0001 versus 0 s and p<0.0001 versus 0.25 J/cm²). The length of action potential block increased exponentially with exposure according to:

t_{AP}=0.1e^{3.54*E},R²=0.992 Equation 1

where t_{AP} is the length of the action potential and E is the exposure.

The average time to recovery for a neuron exposed to 0.90 J/cm² was 2.75 s, with a maximum, average recovery time of 11.1 s following a 0.90 J/cm² exposure (see FIG. 16B). Recovery time increased linearly with exposure according to:

t_{R}=13.5*E−1.1,R²=0.98 Equation 2

where t_{R} is recovery time.

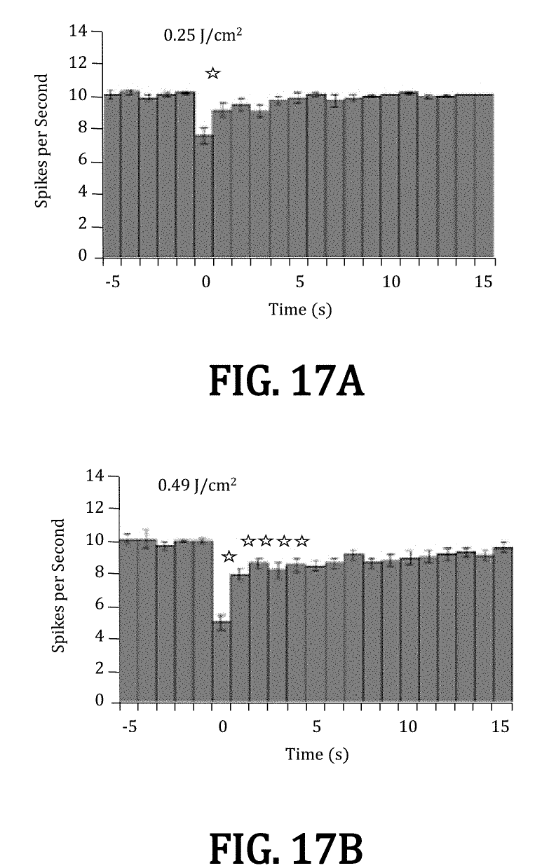

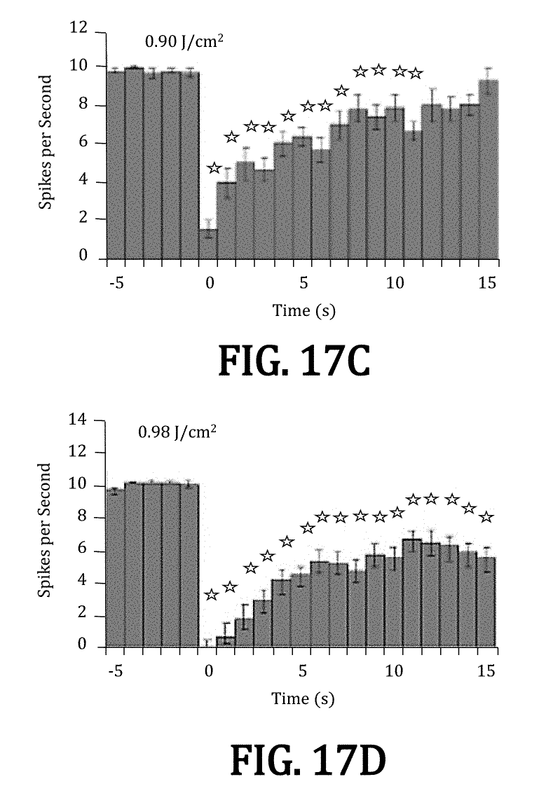

The histograms of FIGS. 17A-17D graphically illustrate an average number of spikes per second following a short infrared laser pulse exposure (0.25 J/cm², 0.49 J/cm², 0.90 J/cm², and 0.98 J/cm², respectively). Significant reductions in the number of spikes per second were observed immediately following exposure, and recovery to 10 spikes per second was observed in all but the 0.98 J/cm² exposure. The inter-pulse intervals (having an expected value of 0.1 s) at 5 s and 15 s, post short infrared laser pulse, demonstrated improved consistency of action potential at 15 s and increased, inconsistently, with radiant exposure (graphically illustrated in FIG. 18).

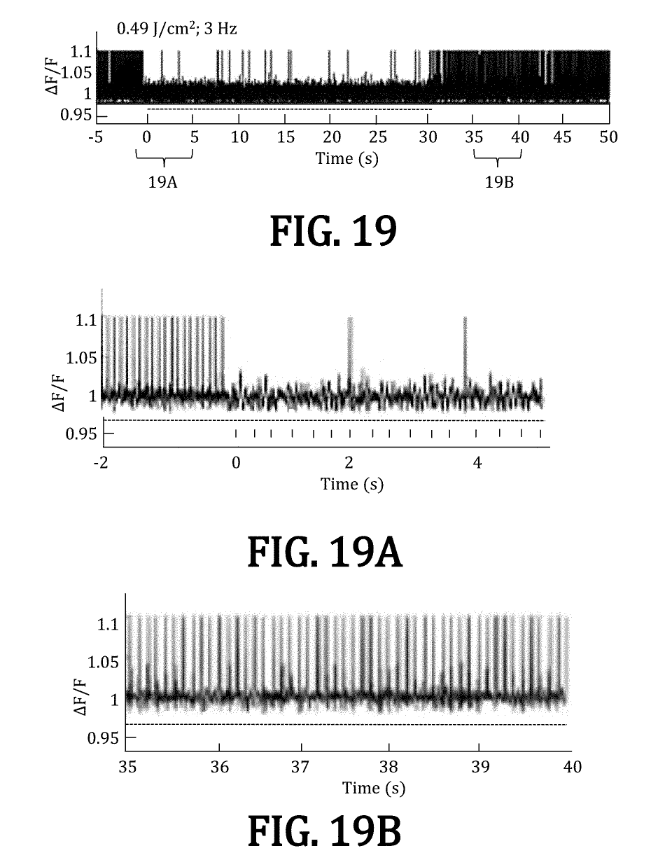

Delivering repeated 0.49 J/cm² short infrared laser pulses sustained a 30 s action potential block. FIG. 19 includes a representative QuasAr2 fluorescence spike plot that demonstrates consistent action potential generation prior to exposure, decreased action potential activity during repeated exposure of 0.49 J/cm2 at 3 Hz (illustrated by the dashed line between 0 s and 30 s), and followed with the return of consistent action potential generation after exposure is removed. FIGS. 19A and 19B are enlargements of the respective portions indicated in FIG. 19. In FIG. 19A, consistent action potentials are displayed from −2 s to 5 s, prior to exposure to the infrared light. In FIG. 19B, the action potential recovery after ceasing exposure is shown.

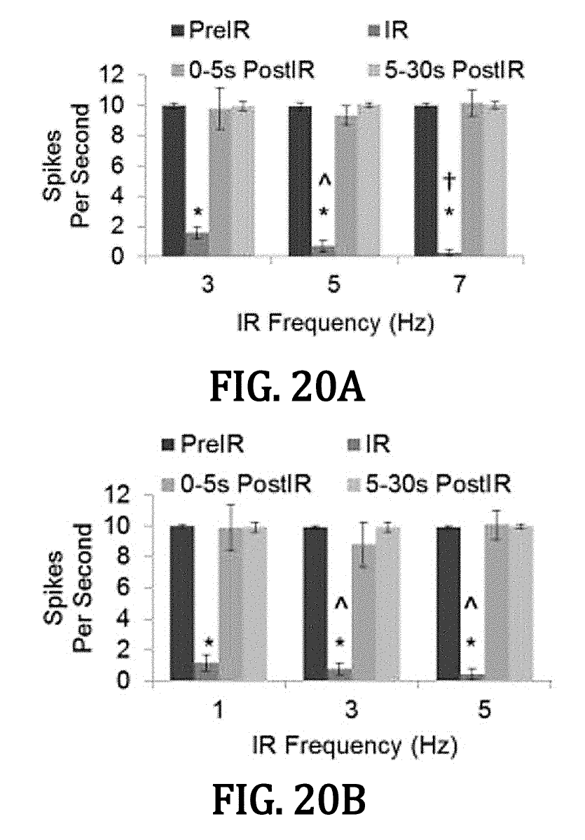

The average spikes per second for the neurons prior to prior to exposure, during exposure, for a time from 0 s to 5 s after exposure, and for a time from 5 s to 30 s after exposure is shown for various exposure frequencies in FIGS. 20A and 20B.

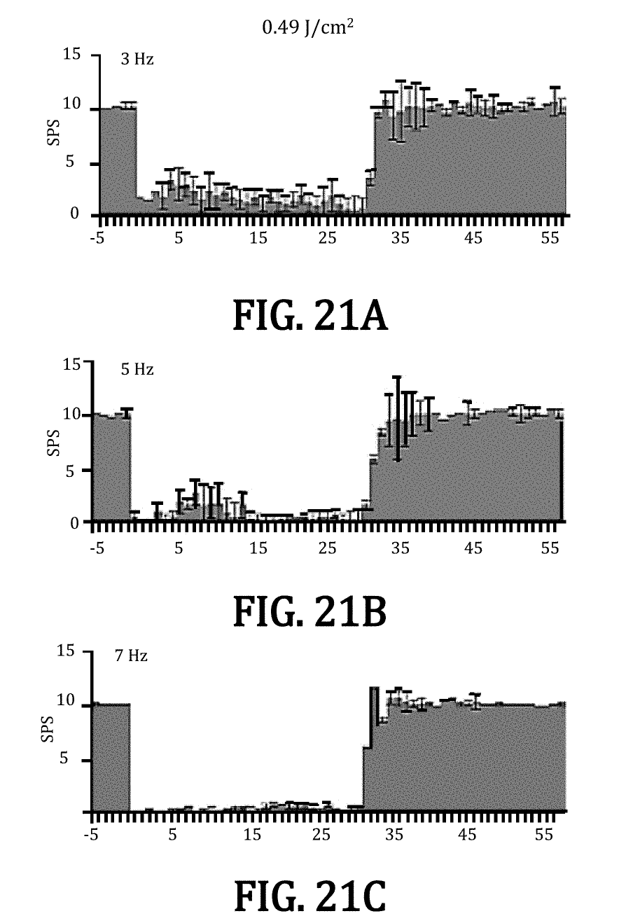

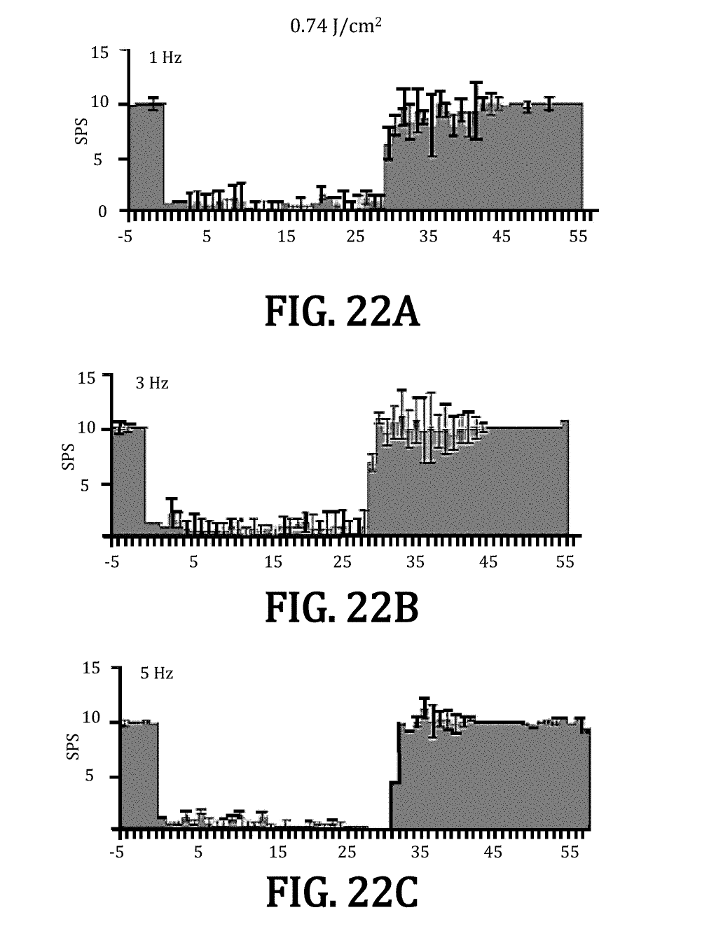

Each of FIGS. 21A-21C demonstrates uniform action potential generation before the short infrared laser pulse, reduced action potential generation during exposure, and recovery after exposure terminated. The average number of action potentials per second was greatly reduced with greater exposure (see FIGS. 22A-22C). For example, in FIG. 21C, 95.4% inhibition was observed with 0.74 J/cm² at 5 Hz; in FIG. 19C, 97.4% inhibition was observed with 0.49 J/cm² at 7 Hz. A significant reduction (p<0.001) in the spikes per second was observed with increasing pulse frequency from 1 Hz to 3 Hz, 5 Hz, or 7 Hz.

Transient, infrared inhibition of excitable cells according to various embodiments of the present invention is described herein. Such embodiments may provide benefit as a research tool by enabling basic science studies of action potential generation, propagation, and inhibition. Additionally, infrared inhibition could be used to study neuronal networks and assess neuron behavior following inhibition or damage. Finally, embodiments of the present invention may be useful for man-machine interfaces or neural-integrated prosthetics. Neuron inhibition occurs naturally in concert with neuron activation to achieve desired motions, thoughts, and actions. Infrared radiation mediated inhibition of neurons may be a surrogate for natural inhibition in brain controlled robots or in neural-integrated prosthetics.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. A method of inhibiting action potentials in excitable cells, the method comprising:
exposing the excitable cells to a pulse of infrared light having a wavelength ranging from 700 nm to about 3 μm and having a radiant exposure at a surface of the excitable cells ranging from 1 μJ/cm² to 1000 J/cm².

2. The method of claim 1, wherein a duration of the pulse of infrared light ranges from 1 μs to 100 ms.

3. The method of claim 1, wherein repetition frequency of the pulse of infrared light ranges from about 100 Hz to 0 Hz or a single pulse.

4. The method of claim 1, wherein the pulse of infrared light is delivered to the excitable cells by way of fiber optics.

5. The method of claim 1, wherein a source of the pulse of infrared light is free-beam optics, fiber optics, or digital micro-mirrors.

6. The method of claim 1, wherein the infrared pulse exposure is generated from a packet of short pulses with durations ranging from about 1 fs to about 1 ms with a repetition frequency ranging from about 10 Hz to 100 GHz.

7. The method of claim 1, wherein the infrared source consists of multiple wavelengths of infrared energy or a broadband light source containing infrared wavelengths.

8. The method of claim 1, wherein the radiant exposure is configured to produce a thermal rise of at least 1° C./ms proximate to the surface of the excitable cells.

9. A system configured to inhibit action potentials in excitable cells according to the method of claim 1, the system comprising:
a radiant source configured to emit the infrared light;
at least one timer configured to design pulses of the infrared light according to a desired pulse sequence;
at least one delivery system configured to direct the pulses of infrared light to the excitable cells; and
a controller configured to control the radiant source, the at least one time, the at least one delivery system, or a combination thereof.

10. The system of claim 8, wherein the delivery system includes a fiber optic cable.

11. A method of inhibiting action potentials in excitable cells, the method comprising:
exposing the excitable cells to a pulse of infrared light having a wavelength ranging from 700 nm to about 3 μm and having a radiant exposure configured to produce a thermal rise of at least 1° C./ms proximate to a surface of the excitable cells.

12. The method of claim 11, wherein a duration of the pulse of infrared light ranges from 1 μs to 100 ms.

13. The method of claim 11, wherein repetition frequency of the pulse of infrared light ranges from about 100 Hz to 0 Hz or a single pulse.

14. The method of claim 11, wherein the pulse of infrared light is delivered to the excitable cells by way of fiber optics.

15. The method of claim 11, wherein a source of the pulse of infrared light is free-beam optics, fiber optics, or digital micro-mirrors.

16. The method of claim 11, wherein the infrared pulse exposure is generated from a packet of short pulses with durations ranging from about 1 fs to about 1 ms with a repetition frequency ranging from about 10 Hz to 100 GHz.

17. The method of claim 11, wherein the infrared source consists of multiple wavelengths of infrared energy or a broadband light source containing infrared wavelengths.

18. The method of claim 1, wherein the radiant exposure ranges from 1p/cm² to 1000 J/cm².
